(19) United States
(12) Patent Application Publication
(10) Pub. No.: US 2025/0346435 A1
(43) Pub. Date: Nov. 13, 2025

(54) APPARATUS FOR STERILIZING PLASTICS MATERIAL PREFORMS AND METHOD THEREFOR

(71) Applicant: KRONES AG, Neutraubling (DE)

(72) Inventors: Florian GELTINGER, Donaustauf (DE); Juergen SOELLNER, Beratzhausen (DE)

(21) Appl. No.: 19/200,314

(22) Filed: May 6, 2025

(30) Foreign Application Priority Data
May 8, 2024 (DE) 10 2024 112 996.4

(51) Int. Cl. B65G 45/10; A61L 2/08; A61L 2/10; A61L 2/20; A61L 2/26; B65G 29/00

(52) U.S. Cl. CPC B65G 45/10; A61L 2/087; A61L 2/10; A61L 2/20; A61L 2/26; B65G 29/00; A61L 2202/11; A61L 2202/23; B65G 2201/0235

(57) ABSTRACT

An apparatus for treating piece goods has a transport device for transporting the goods and at least one application device for sterilizing the goods that are transported by the transport device, to sterilize them with radiation and/or to apply a flowable sterilization agent. The transport device has a first transport unit rotatable with respect to an axis of rotation which has a plurality of first holding devices for holding the goods. The transport device has a second transport unit rotatable with respect to the axis of rotation which has a plurality of second holding devices, each arranged in a second level perpendicular with respect to the axis of rotation. The transport device has at least one first lifting device for displacing a predetermined first portion of the first holding devices with respect to a predetermined second portion of the first holding devices in the direction of the axis of rotation.

### CROSS REFERENCE TO RELATED APPLICATION

This application claims priority from German Patent Application Serial No. 10 2024 112 996.4, Filed May 8, 2024, the contents of which are incorporated herein by reference.

### BACKGROUND OF THE INVENTION

The present invention relates to an apparatus and a method for treating and in particular sterilizing piece goods, and in particular plastics material preforms or containers, in particular plastics material containers.

It has long been known in the prior art to transform heated plastics material preforms into plastics material containers. It has also long been known in the prior art to sterilize such formed plastics material containers, for example if they are subsequently to be filled with certain beverages.

However, it has recently also become known to sterilize the heated plastics material preforms and then transform them into containers, for example in a stretch blow molding machine. The advantage of this procedure is that the comparatively small plastics material preforms, and not significantly larger containers or bottles, need to be sterilized.

For this purpose, it is known that the plastics material preforms are introduced into a treatment room and, within this treatment room, are exposed to a sterilization medium such as hydrogen peroxide.

However, these procedures have the disadvantage of being limited in their efficiency. For example, it would not be possible to transport the plastics material preforms through the treatment room at a significantly increased speed since the time required for sterilization would then not be sufficient.

DE 10 2022 212 5620 A1 discloses a treatment system with a treatment carousel with a first and a second treatment level. The containers are treated in the treatment carousel on a first level, and are subsequently removed from the carousel and then conveyed to a second level and transferred to the carousel on this second level to be treated again there. In this way, different treatment times can be realized.

EP 0 998 418 B1 describes a multi-level apparatus for transporting containers. This apparatus has a plurality of gripping elements which can be displaced vertically during one rotation of the apparatus in order to lift the containers to different levels. There can be a treatment of the containers on the individual levels.

The present invention is therefore based on the object of providing an apparatus and a method which improve the efficiency and output of such sterilization systems.

### SUMMARY OF THE INVENTION

An apparatus according to the invention for treating and in particular sterilizing piece goods, and in particular plastics material preforms or (plastics material) containers, has a transport device that is suitable and intended for transporting the piece goods along a circular transport path. In addition, the apparatus has at least one application device which is suitable and intended to sterilize the piece goods transported by the transport device, in particular to sterilize them with radiation and/or to apply a flowable medium and in particular a sterilization agent.

Furthermore, the transport device has a first transport unit rotatable with respect to an axis of rotation which has a plurality of first holding devices which are each suitable and intended for holding the piece goods and in particular plastics material preforms or (plastics material) containers. These holding devices are arranged equidistantly (one behind the other) in a first level running perpendicular with respect to the axis of rotation (and in particular in a circumferential direction defined with respect to the axis of rotation).

Furthermore, the transport device has a second transport unit rotatable with respect to the axis of rotation which has a plurality of second holding devices which are each suitable and intended for holding the piece goods and in particular the plastics material preforms or (plastics material) containers and which are arranged equidistantly (one behind the other) in a second level perpendicular with respect to the axis of rotation, wherein the second level is offset in the direction of the axis of rotation with respect to the first level.

An equidistant arrangement of the holding devices is understood in particular to mean that they have a constant pitch or are at equal distances from one another in the circumferential direction.

According to the invention, the transport device has at least one first lifting device which is suitable and intended to displace a predetermined first portion of the first holding devices with respect to a predetermined second portion of the first holding devices in the direction of the axis of rotation.

Preferably, therefore, said lifting can be carried out on the same transport device which also has the several transport units arranged in particular one above the other.

It is therefore proposed within the scope of the invention that a certain portion of the first holding devices (in particular with the plastics material preforms arranged thereon) is displaced in the direction of the axis of rotation, i.e. in particular raised or lowered. In this way, as explained in more detail below, it is possible to fill several levels of holding devices with plastics material preforms, or in general piece goods, in order to thus create the possibility of sterilizing or, in general, treating more plastics material preforms, in particular at the same time.

Particularly preferably, said first lifting device is therefore assigned to a first portion of the first holding devices. For example, it is possible that the first lifting device is assigned to a first third of the first holding devices.

As described in more detail below, a treatment and a transport of the piece goods and in particular plastics material preforms therefore takes place on at least two levels. Preferably, an input and output take place, in particular via a holding device (which is in particular a neck handling clamp) on one level. The piece goods are then moved up or down (i.e. along the axis of rotation).

Preferably, the plastics material preforms or piece goods in general are usually displaced while they are being moved in the transport direction or along the transport path. Preferably, the piece goods are moved continuously in the transport direction and/or are continuously movable in the transport direction.

Preferably, the piece goods and in particular plastics material preforms or (plastics material) containers are only transferred to a further transport device, such as a discharge starwheel, at the earliest on every second or subsequent round.

Preferably, the piece goods and in particular plastics material preforms or (plastics material) containers are treated in both levels and in particular gassed with gaseous sterilization agent via nozzles. Preferably, the piece goods and in particular plastics material preforms or (plastics material) containers are moved in groups.

The vertical movement, or lifting movement, in the direction of the axis of rotation is preferably carried out electrically, pneumatically, or cam-controlled.

In a further advantageous embodiment, the number of first holding devices is equal to the number of second holding devices. In particular, the number of holding devices of the second transport unit is therefore equal to the number of holding devices of the first transport unit.

Preferably, the movements of said transport units are coupled in the transport direction. This means that in this case, the at least two, but generally all, transport units move at the same speed in the same direction of rotation. In a preferred embodiment, the movements of all transport units in the transport device are coupled to one another.

Particularly preferably, the second holding devices (i.e. the holding devices of the second transport unit) are arranged exactly above or below the first holding devices (i.e. the holding devices of the first transport unit). This means, for example, that a plastics material preform that is held by a second holding device is geometrically directly above or below a plastics material preform that is held by one of the first holding devices. However, at least one application device (in particular for treating the lower plastics material preform) is preferably provided between these two plastics material preforms.

Arranged right or directly above one another means that the geometric longitudinal axes or axis of rotation of these two plastics material preforms coincide. In other words, in a projection in the direction of the axis of rotation of the transport device, all second holding devices (of the second transport unit) are arranged exactly above or below the particular first holding devices (of the first transport unit).

The piece goods are particularly preferably plastics material preforms or plastics material containers. Particularly preferably, the second holding devices are arranged exactly above the first holding devices in the longitudinal direction of the plastics material preforms.

Particularly preferably, the above-mentioned first level and the second level are spaced apart from each other by at least the length of a piece good and in particular of a plastics material preform. Particularly preferably, the levels are spaced further apart than the longitudinal extent of the plastics material preforms. However, the levels are particularly preferably spaced apart from one another by less than three times the length and preferably less than twice the length of the piece goods, in particular less than three times the length and in particular less than twice the length of the plastics material preforms. These dimensions allow for effective sterilization of the plastics material preforms on the one hand, and on the other hand the installation space for the apparatus can be kept small.

Particularly preferably, the holding devices are gripping devices, and in particular gripping clamps. Particularly preferably, these holding devices or gripping clamps are suitable and intended to hold the plastics material preforms in a mouth region and in particular below their bearing rings. Particularly preferably, the gripping clamps are controllable gripping clamps.

Preferably, therefore, at least one of the transport units is designed segmented, wherein each segment being assigned one of the plurality of holding devices. Preferably, several and particularly preferably all, transport units are designed segmented in this way.

Preferably, at least three such segments are provided. In this way, two of the segments can remain at a constant height while loading the plastics material preforms and unloading the plastics material preforms, and the third segment or further segments can be displaced in height or along the axis of rotation.

In a further advantageous embodiment, the apparatus has a housing within which a treatment chamber is designed for treating and in particular sterilizing the piece goods and in particular the plastics material preforms. Particularly preferably, the above-mentioned transport device is arranged within this treatment room. This treatment room is preferably a clean room that is shielded from a (non-sterile) environment.

Particularly preferably, this housing creates an airtight or substantially airtight closure of the treatment room. Substantially means that any existing airlocks through which plastics material preforms are supplied or discharged are disregarded.

In a further advantageous embodiment, the holding devices and in particular gripping clamps are made of a material which is resistant to sterilization media. Particularly preferably, the holding devices are arranged equidistant from one another in the transport direction. Particularly preferably, the first portion of the first holding devices is equal to the second portion of the first holding devices.

Particularly preferably, the total number of holding devices of each transport unit is divisible by three.

In a preferred embodiment, the first lifting device is suitable and intended to displace a predetermined first portion of the second holding devices with respect to a predetermined second portion of the second holding devices in the direction of the axis of rotation.

This means that not the entirety of the first holding devices are necessarily displaced, but only a certain proportion. Particularly preferably, these holding devices are arranged one behind the other in the circumferential direction of the transport device. In this way, a segment with a plurality of holding devices is preferably displaced or can be accordingly displaced.

Preferably, this displacement of the predetermined first portion of the second holding devices is coupled to the displacement of the predetermined first portion of the first holding devices.

This means that preferably, a first segment with the first portion of the first holding devices and a corresponding segment with a first portion of the second holding devices are displaced along the axis of rotation, wherein preferably these segments lie one above the other in the direction of the axis of rotation. Particularly preferably, the portions of the first holding devices that are displaced and the portions of the second holding devices that are displaced are identical or of the same size.

The portion of the first holding devices could also be individual holding devices. Preferably, however, there are several holding devices, preferably at least two, preferably at least three, preferably at least four, preferably at least six and preferably at least ten holding devices.

Preferably, however, these are groups of holding devices arranged directly one behind the other in the transport device.

In this way, it is possible to equip or load the individual transport units one after the other with plastics material preforms or in general with piece goods.

In a further advantageous embodiment, the transport device has a supply region in which piece goods and in particular plastics material preforms can be supplied to the transport device, wherein the piece goods can be supplied in a supply level with respect to which the axis of rotation is perpendicular.

Furthermore, the transport device preferably has a discharge region in which the piece goods and in particular plastics material preforms can be discharged from the transport device, wherein the piece goods can be supplied in a discharge region with respect to which the axis of rotation is perpendicular.

In a preferred embodiment, the supply level and the discharge level lie on substantially the same level or at substantially the same height with respect to the axis of rotation. Substantially the same height is understood here to mean that the heights of these levels do not differ by more than 5 cm, preferably by no more than 3 cm and preferably by no more than 1 cm and preferably by no more than 0.5 cm.

Preferably, the supply level and the discharge level remain constant, i.e. the piece goods are always supplied and discharged at the same level.

Particularly preferably, therefore, the supply level and the discharge level substantially coincide, wherein the supply region is offset from the discharge region in the circumferential direction of the first transport device.

In a further advantageous embodiment, the transport device has a third transport unit rotatable with respect to the axis of rotation which has a plurality of third holding devices which are each suitable and intended for holding the piece goods and in particular plastics material preforms or containers and which are preferably arranged equidistantly in a third level perpendicular with respect to the axis of rotation. This third level is offset in the direction of the axis of rotation from the first level and the second level.

In a preferred embodiment, a distance between the third level and the second level is equal to the distance between the second level and the first level. In a further advantageous embodiment, the number of third holding devices is equal to the number of first holding devices and the number of second holding devices.

Preferably, the third holding devices also lie exactly above the first and/or second holding devices.

Particularly preferably, the transport circuits of the individual holding devices of the first and second and preferably also of the third transport unit correspond to one another.

It is to be noted that the apparatus described here for treating piece goods can also comprise two or more of the transport devices described here. Preferably, at least two and preferably at least three such transport devices are provided. Preferably, all of these transport devices are arranged in the treatment room described above.

In a further preferred embodiment, the transport device can also have more than three transport units.

In a further preferred embodiment, the transport device has a second lifting device which is suitable and intended to displace the predetermined second portion of the first holding devices relative to the predetermined first portion of the first holding devices in the direction of the axis of rotation.

The lifting device could, for example, be a guide cam that interacts with several cam rollers that can raise and lower individual segments. In this case, a lifting device is understood to be the combination of this guide cam with a specific cam roller and the corresponding carrier of the cam roller, which can then raise and lower the respective holding devices. Therefore, even in an embodiment with a second guide roller, a second lifting device would be provided. Preferably, the lifting devices also each have guide devices which are suitable and intended for guiding the displacement movement along the axis of rotation.

In a further preferred embodiment, the transport device has a third lifting device which is suitable and intended to displace a predetermined third portion of the first holding devices relative to the predetermined first portion of the first holding devices and/or relative to the predetermined first portion of the second holding devices in the direction of the axis of rotation. This third lifting device can also be a lifting device operated electrically, pneumatically, hydraulically, or by a guide cam.

In a further preferred embodiment, more than three, or n, lifting devices are provided.

In a preferred embodiment, therefore, a first transport unit with first holding devices, a second transport unit with second holding devices and a third transport unit with third holding devices are provided. These first, second and third holding devices preferably each have first, second and third portions of holding devices.

Therefore, the first lifting unit is particularly preferably assigned to the first portion of the first holding devices, the second lifting unit to the second portion of the first holding devices, and the third lifting unit to a third portion of the first holding devices. Preferably, these first, second and third portions are each of equal size.

In this way, it is always possible during ongoing operation, for example, to supply plastics material preforms via a first portion of the holding devices or a first segment held constant along the axis of rotation, and to discharge plastics material preforms via a further segment, for example a third segment, held constant correspondingly along the axis of rotation.

A segment lying between these two first and third mentioned segments can, however, be displaced in order to thereby enable the complete loading of the transport device, in particular with plastics material preforms or containers.

In a further preferred embodiment, the second lifting device is suitable and intended to displace a predetermined second portion of the second holding devices relative to a predetermined first portion of the second holding devices in the direction of the axis of rotation.

Particularly preferably, this displacement of the predetermined second portion of the second holding devices is coupled to the displacement of the predetermined second portion of the first holding devices.

Overall, one can imagine the composition of the two or preferably three transport units in such a way that individual segments can be raised or lowered relative to the remaining segments, similar to pieces of a cake.

In a further preferred embodiment, the application device has a plurality of application elements and in particular application nozzles which are preferably each assigned to the holding devices and which are particularly preferably suitable and intended to sterilize and/or apply a sterilization medium to the inner surfaces of the plastics material preforms.

Preferably, the apparatus has a plurality of application segments, each of which is displaceable together with the particular portions of holding devices with respect to the axis of rotation. Particularly preferably, several of such application segments are assigned to each transport unit, wherein preferably at least one such application segment is assigned to each portion of the particular holding devices of each transport unit. Preferably, the application segments are of the same type or are designed as identical parts.

Preferably, therefore, at least one device of the application device (and in particular at least one of the above-mentioned application segments) is displaceable along the axis of rotation. Preferably, the movement of this device, i.e. in particular of the application segment, is coupled to the movement of at least one portion of the holding devices.

In a further preferred embodiment, these application devices and/or application segments are preferably arranged above the holding devices assigned to them. In this way, a treatment of the plastics material preforms transported with the mouths facing upwards is enabled.

Particularly preferably, the transport device is arranged in an in particular closed housing.

Particularly preferably, the transport device and/or the apparatus has a (central) supply unit for applying a sterilization medium to the plastics material preforms. Particularly preferably, the sterilization medium is hydrogen peroxide or peracetic acid.

Also preferably, the transport device and/or the apparatus comprises a (central) radiation-generating device, in particular a radiation source, for sterilizing the plastics material preforms with radiation. The radiation is particularly preferably electron beams and/or ultraviolet radiation.

In a further preferred embodiment, the apparatus has a rotary distributor. This rotary distributor is used to distribute the sterilization medium from a stationary source to the particular carriers and in particular to the individual application devices, wherein this transfer is intended to be possible in particular also during movement of the transport units.

In a further preferred embodiment, the apparatus has a supply starwheel or a supply wheel which feeds the plastics material preforms to the transport device.

In a further preferred embodiment, the apparatus has a discharge starwheel or a discharge wheel which discharges the plastics material preforms from the transport device. Particularly preferably, the transport device is arranged together with the supply starwheel and the discharge starwheel in a housing.

In a further preferred embodiment, the apparatus has a plurality of carrier segments extending along a circular segment-shaped line, each of which is suitable and intended for jointly carrying a plurality of holding devices. Preferably, each of the carrier segments is suitable and intended for carrying one of the portions of the holding devices that were referred to above.

In a further preferred embodiment, the radiation is provided by at least one radiation-generating device, in particular radiation source, as electron radiation and/or ultraviolet radiation, and/or the radiation is applied by the application device via one, preferably a plurality of, application elements, in particular a beam finger and/or a surface radiator.

The apparatus preferably contains at least one, preferably several, radiation-generating apparatuses, in particular an electron emitter and/or an ultraviolet (UV) emitter.

Preferably, the application device is suitable for applying radiation to the plastics material preforms and thereby sterilizing them.

The plastics material preforms are preferably sterilized using electron beams. The application device has at least one application element, in particular one, preferably several, surface radiators, which are preferably arranged along the transport path of the plastics material preforms and are preferably directed onto their outer surfaces. The at least one surface radiator is preferably arranged stationary relative to the transport device; preferably it is suitable for sterilization, in particular of the plastics material preforms, from the outside.

The application device has at least one application element, in particular a beam finger, preferably several beam fingers, which can be inserted into the plastics material preforms and are preferably inserted into the plastics material preforms. Preferably, the beam finger, preferably the plurality of beam fingers, can be moved along with the transport device, and preferably they are moved along with the transport device and/or the plastics material preforms. Preferably, the beam fingers are suitable and/or intended for internal sterilization. For this purpose, they can preferably be immersed in the interior of the plastics material preforms.

Sterilization is preferably carried out in a substep.

A beam finger is understood to be a rod-like body of any shape, preferably insertable into a container opening, in particular a plastics material preform opening.

When sterilizing the plastics material preforms by radiation, the outside and/or inside are preferably sterilized/disinfected. The plastics material preform rotates around its longitudinal axis in front of/around a radiation-generating device so that all surface points on the outside and/or inside of the plastics material preform undergo the most uniform irradiation possible in terms of quality and quantity.

In order to rotate the plastics material preform around its longitudinal axis, it is proposed to integrate a rotation mandrel, preferably several rotation mandrels, onto which the plastics material preform can be placed and is placed, in particular from below. The mandrels are rotated about their longitudinal axis, at least in the region of the electron source, in order to irradiate the outer shell of the plastics material preform as evenly as possible.

The application device preferably has, as application elements, a plurality of beam fingers which can be inserted into the plastics material preforms in order to apply radiation, in particular electron radiation and/or ultraviolet radiation, to them.

Particularly preferably, electron acceleration devices are provided and/or exit windows are also provided for the exit of the electron beams, which are preferably made of titanium.

In a further preferred embodiment, the sterilization can be carried out as an external and/or internal sterilization, in particular on the same transport device, and/or an external and/or internal sterilization can be carried out on at least two, preferably a plurality of, different transport devices, in particular one after the other in terms of time and/or location.

The application device preferably treats, in particular sterilizes, an outer surface of the plastics material preforms.

For this purpose, a surface radiator is particularly preferably provided which directs radiation, and in particular electron radiation and/or ultraviolet radiation, onto the outer surface of the plastics material preforms. This surface radiator can be arranged on a housing, in particular of the apparatus, in such a way that the radiation strikes the plastics material preforms through an opening in the housing.

The application device preferably treats, in particular sterilizes, an inner surface of the plastics material preforms.

For this purpose, a beam finger is particularly preferably provided which directs radiation, in particular electron radiation and/or ultraviolet radiation, onto the inner surface of the plastics material preforms. This beam finger can be arranged on a housing, in particular of the apparatus, in such a way that the radiation strikes the plastics material preforms through an opening in the housing. For example, radiation-generating devices, in particular electron beams and/or ultraviolet (UV) beams, can be introduced into the containers.

Particularly preferred is the sterilization of both the external and internal surfaces of the plastics material preforms.

The application device with the application elements for external and/or internal sterilization can be configured jointly or separately.

Preferably, the external and/or internal sterilization of a plastics material preform can be carried out at the same time and location.

Particularly preferably, the external and/or internal sterilization of a plastics material preform can be carried out temporally one after the other.

Particularly preferably, the external and/or internal sterilization of a plastics material preform can be carried out locally one after the other.

It is conceivable to carry out external and/or internal sterilization on different transport devices and/or holding devices.

Preferably, the plastics material preforms are exposed to a flowable medium temporally before the outer surface and/or the inner surface of the plastics material preforms are treated with radiation. In particular, the plastics material preform is preferably first pre-cleaned with a flowable medium and then sterilized/disinfected with electron and/or ultraviolet radiation.

This allows particularly good sterilization/disinfection results to be achieved.

Particularly preferably, each carrier segment carries at least three, preferably at least four, preferably at least five, preferably at least six, preferably at least eight and preferably at least ten holding devices. Particularly preferably, these holding devices can be individually removed from this carrier segment and/or individually mounted on this carrier segment.

The present invention is further directed to a transport device for transporting plastics material preforms or (plastics material) containers along a circular transport path, wherein the transport device comprises a first transport unit rotatable with respect to an axis of rotation, which has a plurality of first holding devices, each of which is suitable and intended for holding the plastics material preforms or containers and which are arranged equidistantly in a first level perpendicular with respect to the axis of rotation.

Furthermore, the transport device has a second transport unit rotatable with respect to the axis of rotation which has a plurality of second holding devices which are each suitable and intended for holding the plastics material preforms or plastics material containers and which are arranged equidistantly in a second level perpendicular with respect to the axis of rotation, wherein the second level is offset in the direction of the axis of rotation relative to the first level, and wherein an offset of the second level relative to the first level in the direction of the axis of rotation is greater than a length in this direction of the axis of rotation of the plastics material preforms or containers to be transported.

According to the invention, the transport device has at least one first lifting device which is suitable and intended to displace a predetermined first portion of the first holding devices relative to a predetermined second portion of the first holding devices in the direction of the axis of rotation, and furthermore the transport device has a second lifting device which is suitable and intended to displace the predetermined second portion of the first holding devices relative to the predetermined first portion of the first holding devices in the direction of the axis of rotation.

Particularly preferably, the transport device is configured in the manner described above. This means that the features described above with regard to the transport device of the apparatus can also be applied to the transport device described here.

Preferably, this transport device is suitable for transporting the plastics material preforms and/or containers during a predetermined treatment, in particular a sterilization. However, it would also be possible for this treatment to be a heating process, a rinsing process, an inspection process of the plastics material preform, or the like.

Particularly preferably, the two lifting devices have electric drives. However, pneumatic or hydraulic drives or drives using guide cams would also be conceivable.

The present invention is further directed to a method for treating and in particular sterilizing piece goods and in particular plastics material preforms or (plastics material) containers, wherein a transport device transports the piece goods and in particular plastics material preforms or (plastics material) containers along a circular transport path, and at least one application device applies a flowable sterilization agent and/or sterilizing radiation to the piece goods and in particular plastics material preforms or plastics material containers transported by the transport device.

Furthermore, the transport device has a first transport unit rotatable with respect to an axis of rotation which has a plurality of first holding devices (or on which these holding devices are arranged), which each hold the piece goods and in particular plastics material preforms or (plastics material) containers and which are arranged equidistantly in a first level perpendicular with respect to the axis of rotation (in particular in the circumferential direction).

Furthermore, the transport device has a second transport unit rotatable and/or rotating with respect to the axis of rotation which has a plurality of second holding devices which each hold the piece goods and in particular the plastics material preforms or (plastics material) containers and which are arranged equidistantly in a second level perpendicular with respect to the axis of rotation (and in particular a circumferential direction which runs in this level), wherein the second level is offset in the direction of the axis of rotation with respect to the first level. In particular, these two levels are parallel to each other.

According to the invention, the transport device has at least one first lifting unit which at least temporarily displaces a predetermined first portion of the first holding devices relative to a predetermined second portion of the first holding devices in the direction of the axis of rotation.

Particularly preferably, the transport time for all piece goods using the transport device is the same. This is important because it is thereby ensured that the exposure time or sterilization time is kept the same for all plastics material preforms.

Particularly preferably, the rotations of the transport units are coupled to one another.

In a further preferred embodiment, the piece goods and in particular the plastics material preforms and/or containers are supplied to the transport device in a supply level to which the axis of rotation is perpendicular.

Furthermore, the piece goods and in particular the plastics material preforms or plastics material containers are discharged by the transport device in a discharge level. The axis of rotation is also perpendicular to this discharge level. Particularly preferably, the supply level and the discharge level (substantially) coincide. This means that the piece goods, and in particular plastics material preforms or containers, are supplied in at the same level or the same height at which they are discharged.

In a further preferred method, each plastics material preform and/or each plastics material container is transported through a transport angle that is greater than 360°, preferably greater than 720°. This means that piece goods and in particular plastics material preforms or containers are preferably transported by more than 1 rotation and preferably by more than 2 rotations.

In a further preferred embodiment, each piece good and in particular each plastics material preform or each container is displaced at least once in the direction of the axis of rotation in a first displacement direction and at least once in the direction of the axis of rotation in a second displacement direction which is opposite the first displacement direction.

In a further preferred method, each piece good and in particular each plastics material preform and/or each container is displaced at least twice in the direction of the axis of rotation in a first displacement direction. Preferably, the transport device is loaded with the piece goods and in particular the plastics material preforms or containers in this way.

Particularly preferably, said displacement also takes place in the second direction, which is opposite to the first direction, at least once, preferably at least twice.

In a further preferred method, portions of plastics material preforms and/or plastics material containers arranged one behind the other in the transport direction are displaced together along the axis of rotation. In a further preferred method, plastics material preforms or containers arranged one above the other are moved together along the axis of rotation.

Preferably, at least one of the segments is displaced along the axis of rotation during a production or during a treatment process. Preferably, however, only exactly one of the segments is displaced along the axis of rotation at a predetermined point in time or time period.

In a further preferred embodiment, the treatment device has at least one transport device which is suitable and intended to change a distance between successive plastics material preforms (also referred to as a pitch varying starwheel). In a further preferred embodiment, the treatment device has at least two transport devices that are suitable and intended to change a distance between successive plastics material preforms.

Preferably, the treatment device is therefore divided into several groups of treatment elements assigned to the holding devices, such as in particular treatment nozzles. These can be arranged both on the different transport units as well as in different segments. Preferably, there are at least four groups, preferably at least six groups and particularly preferably at least nine groups (in particular at least three groups per transport unit).

In a further preferred embodiment, a control device is provided which is suitable for activating or deactivating individual transport units. It would therefore be possible to feed piece goods and in particular plastics material preforms to only one of the mentioned transport units or to only two of the mentioned transport units. In this way, the storage capacity of the transport unit can be changed.

In a further preferred embodiment, the transport device has a sealing device that seals the transport device from a (non-sterile) environment. This can be a hydraulic seal, for example. For example, so-called water locks could be used for sealing. Such water locks preferably have a circumferential stationary channel filled with water or another liquid, into which a circumferential blade is immersed in order to create the seal.

In a further preferred embodiment, the transport device is arranged inside a clean room.

Preferably, the apparatus has at least one sensor device which is suitable and intended to detect at least one property which is characteristic of a gaseous medium located inside the treatment room. This property can be, for example, a pressure, a temperature or a concentration of sterilization agent.

### BRIEF DESCRIPTION OF THE DRAWINGS

Further advantages and embodiments emerge from the accompanying drawings.
in which:

FIG. 1 shows a schematic representation of a system for producing plastics material containers;

FIG. 2 shows a representation of a transport device according to the invention; and

FIG. 3 shows a representation to illustrate a method according to the invention.

### DETAILED DESCRIPTION OF THE INVENTION

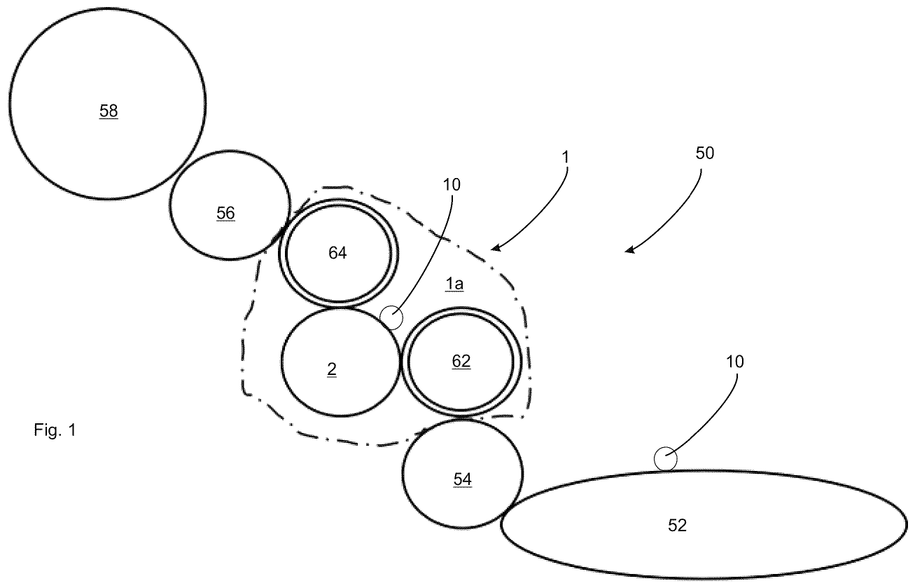

FIG. 1 schematically shows a system 50 for producing plastics material containers. This system has a heating device 52 which heats the plastics material preforms 10. This can be, for example, an infrared oven or a microwave oven. The reference sign 54 denotes a transport device which supplies the heated plastics material preforms to the treatment device 1, which is a sterilization device. Preferably, this transport device 54 is a so-called pitch varying starwheel which can change a distance or pitch between the plastics material preforms.

Reference sign 1 designates the treatment device. The reference sign 1a designates a treatment room within which the treatment and in particular sterilization of the plastics material preforms is carried out. The reference sign 2 denotes the transport device described above, to which plastics material preforms are supplied by a supply device 62 and from which the sterilized plastics material preforms are discharged by a discharge device 64.

The reference sign 56 denotes a further transport device, preferably again in the form of a pitch varying starwheel, which discharges the sterilized plastics material preforms. This further transport device 56 is therefore preferably suitable and intended to change and in particular to increase a pitch between the transported plastics material preforms.

The reference sign 58 denotes a forming device, such as in particular a blow molding machine and in particular a stretch blow molding machine which forms the supplied plastics material preforms into plastics material containers.

Particularly preferably, this forming device is a sterile forming device, i.e. a forming device which has a clean room within which the plastics material preforms are formed into the plastics material containers. Preferably, this clean room is designed channel-like around a transport path of the plastics material preforms.

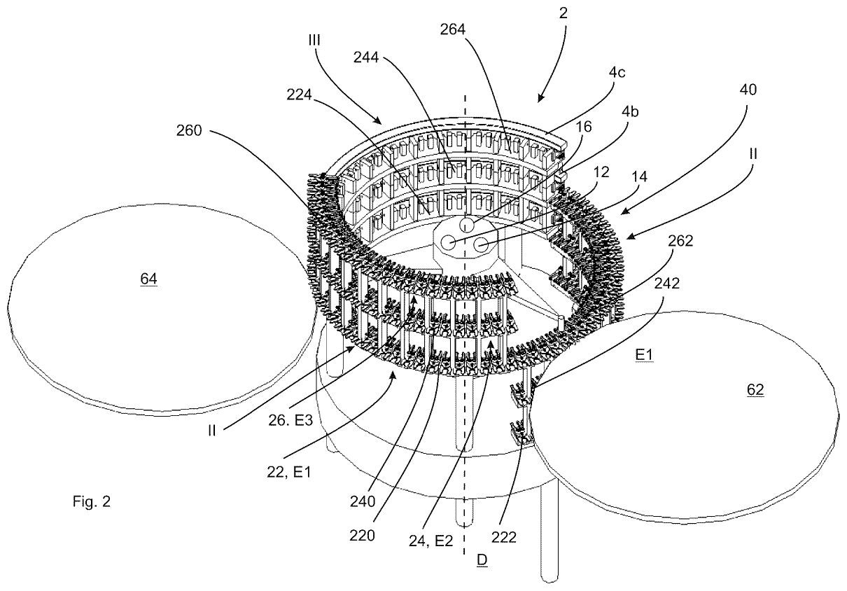

FIG. 2 shows a representation of the transport device 2 according to the invention. This transport device has a first transport unit 22 which is arranged in a first level E1 so as to be rotatable with respect to an axis of rotation D. The reference sign 24 denotes a second transport unit which is also arranged rotatably with respect to the axis of rotation D in a second level E2 which is offset in the direction of the axis of rotation D with respect to the first level E1.

The reference sign 26 denotes a third transport unit which is arranged in a third level E3 so as to be rotatable with respect to an axis of rotation D. The third level E3 is offset from the first level E1 and the second level E2 in the direction of the axis of rotation.

Preferably, the rotational movements of the individual transport units 22, 24, 26 are coupled with each other with respect to the axis of rotation.

The first transport unit 22 has a plurality of first holding devices 220, 222 and 224, each of which is suitable and intended for holding plastics material preforms. More specifically, these are a first portion 220 of first holding devices, a second portion 222 of first holding devices and a third portion 224 of first holding devices. These particular portions can each be displaced together in the direction of the axis of rotation D, but relative to each of the other portions.

The second transport unit 24 has a plurality of second holding devices 240, 242 and 244, each of which is suitable and intended for holding plastics material preforms. More specifically, these are a first portion 240 of second holding devices, a second portion 242 of second holding devices and a third portion 244 of second holding devices. These particular portions of holding devices can also each be moved together in the direction of the axis of rotation, but relative to each of the other portions.

The third transport unit 26 has a plurality of third holding devices 260, 262 and 264, each of which is suitable and intended for holding plastics material preforms. More specifically, these are a first portion 260 of third holding devices, a second portion 262 of third holding devices and a third portion 264 of third holding devices. These particular portions of holding devices can also each be displaced together in the direction of the axis of rotation D, but relative to the respective other portions.

The reference sign 12 designates a first lifting device. This first lifting device serves to displace the first portion 220 of the first holding devices which is arranged on the first transport unit 22, the first portion 240 of the second holding devices which is arranged on the second transport unit 24, and the first portion 260 of the third holding devices which is arranged on the third transport unit 26, in the direction of the axis of rotation D, i.e. to raise or lower it.

The reference sign 14 designates a second lifting device. This second lifting device serves to move the second portion 222 of the first holding devices which is arranged on the first transport unit 22, the second portion 242 of the second holding devices which is arranged on the second transport unit 24, and the second portion 262 of the third holding devices which is arranged on the third transport unit 26, in the direction of the axis of rotation D, i.e. to raise or lower it.

In the situation shown in FIG. 2, these particular second portions are each lowered relative to the first portions in the direction of the axis of rotation D.

The reference sign 16 designates a third lifting device. This third lifting device serves to move the third portion 224 of the first holding devices which is arranged on the third transport unit 26, the second portion 244 of the second holding devices which is arranged on the third transport unit 26, and the third portion 264 of the third holding devices which is arranged on the third transport unit 26, in the direction of the axis of rotation D, i.e. to raise or lower it.

In each case, the first, second and third portions are moved together in the direction of the axis of rotation. The reference signs I-III each designate segments of holding devices that can be displaced together.

The reference signs 4b and 4c designate two application devices which serve to apply a flowable sterilization agent and/or sterilizing radiation to the plastics material preforms (not shown) held by the holding devices.

Preferably, each transport unit 22, 24, 26 is assigned several, here three, application devices. Preferably, the number of application devices per transport unit corresponds to the number of segments.

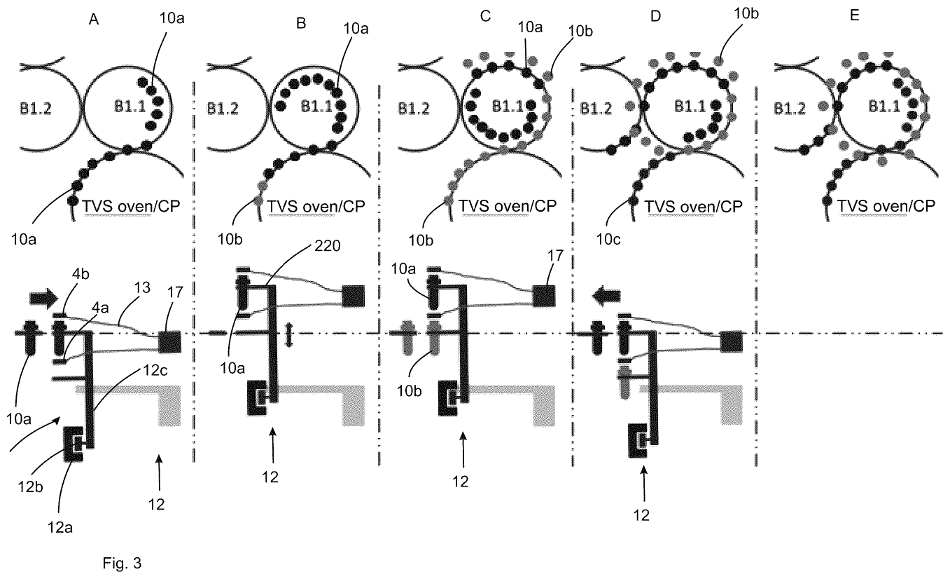

FIG. 3 illustrates the method according to the invention. Five process situations A-E are shown, wherein the process situations A-C relate to a loading process of the transport device 2 with plastics material preforms 10a, 10b, and the process situations D and E relate to an unloading process.

In the process situation A, a first group of plastics material preforms 10a is supplied to the transport device. In this case, these plastics material preforms are each gripped by holding devices 220 of the first portion of the holding devices of the first transport unit. The reference signs 4a and 4b show application devices, wherein the application devices are each assigned to the holding devices 220.

In the process situation B, the holding devices 220 are lifted by the lifting device 12 which here has a guide cam 12a as well as a guide roller 12b and a carrier 12c displaceable in the vertical direction V. The holding devices also lift the application devices 4a and 4b.

In the process situation C, further plastics material preforms 10b are supplied, but now to the subsequent segment whose holding devices have not yet been displaced along the axis of rotation. The mentioned holding devices 220 remain in the upper position here. The upper part of the image of the process situation shows that a second group of plastics material preforms 10b is now supplied to the transport device.

In the process situation D, the holding devices 220 are lowered again so that the originally supplied plastics material preforms can be discharged again by the transport device.

In the process situation E, a third group of plastics material preforms was additionally supplied.

It is to be noted that all features which have been described with reference to the method are also disclosed in a corresponding manner for the apparatus, which means in particular that the corresponding apparatus has devices which are suitable and intended for carrying out the particular methods. Furthermore, features which were described with reference to the apparatus are also correspondingly applicable to the method or methods. This means that the methods are carried out using the corresponding apparatus features.

The applicant reserves the right to claim all features disclosed in the application documents as essential to the invention, provided that they are novel over the prior art individually or in combination. It is also pointed out that features which can be advantageous in themselves are also described in the individual figures. A person skilled in the art will immediately recognize that a particular feature described in a figure can be advantageous even without the adoption of further features from this figure. Furthermore, a person skilled in the art will recognize that advantages can also result from a combination of several features shown in individual or in different figures.

1. An apparatus for treating piece goods, having a transport device which is configured to transport the piece goods along a circular transport path and having at least one application device which is configured to sterilize the piece goods that are transported by the transport device, wherein the transport device has a first transport unit rotatable with respect to an axis of rotation which has a plurality of first holding devices which are each configured to hold the piece goods and which are arranged equidistantly in a first level perpendicular with respect to the axis of rotation,
and wherein the transport device has a second transport unit rotatable with respect to the axis of rotation which has a plurality of second holding devices which are each configured to hold the piece goods and which are arranged equidistantly in a second level perpendicular with respect to the axis of rotation, wherein the second level is offset in the direction of the axis of rotation relative to the first level,
wherein the transport device has at least one first lifting device which is configured to displace a predetermined first portion of the first holding devices relative to a predetermined second portion of the first holding devices in the direction of the axis of rotation.

2. The apparatus according to claim 1,
wherein
the first lifting device is configured to displace a predetermined first portion of the second holding devices relative to a predetermined second portion of the second holding devices in the direction of the axis of rotation.

3. The apparatus according to claim 1,
wherein
the transport device has a supply region in which the piece goods are supplied to the transport device, wherein the piece goods are supplied in a supply level with respect to which the axis of rotation is perpendicular, and the transport device has a discharge region in which the piece goods are discharged from the transport device, wherein the piece goods are discharged in a discharge level with respect to which the axis of rotation is perpendicular.

4. The apparatus according to claim 1,
wherein
the transport device has a third transport unit rotatable with respect to the axis of rotation which has a plurality of third holding devices which are each configured for holding the piece goods and which are arranged equidistantly in a third level perpendicular with respect to the axis of rotation, wherein the third level is offset in the direction of the axis of rotation relative to the first level and the second level, and/or the transport device has a second lifting device which is configured for displacing the predetermined second portion of the first holding devices relative to the predetermined first portion of the first holding devices in the direction of the axis of rotation.

5. The apparatus according to claim 1,
wherein
the transport device has a third lifting device which is configured to displace a predetermined third portion of the first holding devices relative to the predetermined first portion of the first holding devices and relative to the predetermined second portion of the first holding devices in the direction of the axis of rotation.

6. The apparatus according to claim 1,
wherein
the second lifting device is configured to displace a predetermined second portion of the second holding devices relative to a predetermined first portion of the second holding devices in the direction of the axis of rotation.

7. The apparatus according to claim 1,
wherein
the application device has a plurality of application elements which are each assigned to the holding devices.

8. The apparatus according to claim 1,
wherein
the apparatus has a plurality of carrier segments extending along a circular segment-shaped line, each of which is configured for jointly bearing a plurality of holding devices.

9. The apparatus according to claim 1,
wherein
the radiation is provided by at least one radiation-generating apparatus as electron radiation and/or ultraviolet radiation, and/or the radiation is applied by the application device via a, preferably a plurality of, application element(s).

10. The apparatus according to claim 1,
wherein
the sterilization is carried out as an external and/or internal sterilization, and/or an external and/or internal sterilization is carried out on at least two different transport devices.

11. A transport device for transporting plastics material preforms or containers along a circular transport path, wherein the transport device has a first transport unit rotatable with respect to an axis of rotation which has a plurality of first holding devices, each of which is configured for holding the plastics material preforms or containers and which are arranged equidistantly in a first level perpendicular with respect to the axis of rotation,
and wherein the transport device has a second transport unit rotatable with respect to the axis of rotation which has a plurality of second holding devices, each of which is configured for holding the plastics material preforms or containers and which are arranged equidistantly in a second level perpendicular with respect to the axis of rotation, wherein the second level is offset in the direction of the axis of rotation relative to the first level, wherein an offset of the second level relative to the first level is greater than a length in the direction of the axis of rotation of the plastics material preforms or containers to be transported,
wherein
the transport device has at least one first lifting device which is configured for displacing a predetermined first portion of the first holding devices relative to a predetermined second portion of the first holding devices in the direction of the axis of rotation, and wherein the transport device has a second lifting device which is configured to displace the predetermined second portion of the first holding devices relative to the predetermined first portion of the first holding devices in the direction of the axis of rotation.

12. A method for sterilizing piece goods, wherein a transport device transports the piece goods along a circular transport path, and at least one application device applies a flowable sterilization agent and/or sterilizing radiation to the piece goods transported by the transport device,
wherein the transport device has a first transport unit rotatable with respect to an axis of rotation which has a plurality of first holding devices, each of which holds the piece goods and which are arranged equidistantly in a first level perpendicular with respect to the axis of rotation, and wherein the transport device has a second transport unit rotatable with respect to the axis of rotation which has a plurality of second holding devices, each of which holds the piece goods and which are arranged equidistantly in a second level perpendicular with respect to the axis of rotation, wherein the second level is offset in the direction of the axis of rotation relative to the first level,
wherein
the transport device has at least one first lifting device which at least temporarily displaces a predetermined first portion of the first holding devices relative to a predetermined second portion of the first holding devices in the direction of the axis of rotation.

13. The method according to claim 12,
wherein
the plastics material preforms or plastics material containers are supplied to the transport device in a supply level to which the axis of rotation is perpendicular, and the plastics material preforms or plastics material containers are removed from the transport device in a discharge level to which the axis of rotation is perpendicular, and/or each plastics material preform and/or each plastics material container is transported through a transport angle which is greater than 360°, preferably greater than 720°.

14. The method according to claim 12, wherein each plastics material preform is displaced at least once in the direction of the axis of rotation in a first displacement direction and at least once in the direction of the axis of rotation in a second displacement direction which is opposite to the first displacement direction, and/or each plastics material preform and/or each plastics material preform is displaced at least twice in the direction of the axis of rotation in a first displacement direction.

15. The method according to claim 12,
wherein
portions of plastics material preforms and/or plastics material containers arranged one behind the other in the transport direction are displaced together along the axis of rotation, and/or in that plastics material preforms or containers arranged one above the other are displaced together along the axis of rotation.

16. The apparatus according to claim 2,
wherein
the transport device has a supply region in which the piece goods are supplied to the transport device, wherein the piece goods are supplied in a supply level with respect to which the axis of rotation is perpendicular, and the transport device has a discharge region in which the piece goods are discharged from the transport device, wherein the piece goods are discharged in a discharge level with respect to which the axis of rotation is perpendicular.

17. The apparatus according to claim 2,
wherein
the transport device has a third transport unit rotatable with respect to the axis of rotation which has a plurality of third holding devices which are each configured for holding the piece goods and which are arranged equidistantly in a third level perpendicular with respect to the axis of rotation, wherein the third level is offset in the direction of the axis of rotation relative to the first level and the second level, and/or the transport device has a second lifting device which is configured for displacing the predetermined second portion of the first holding devices relative to the predetermined first portion of the first holding devices in the direction of the axis of rotation.

18. The apparatus according to claim 2,
wherein
the transport device has a third lifting device which is configured to displace a predetermined third portion of the first holding devices relative to the predetermined first portion of the first holding devices and relative to the predetermined second portion of the first holding devices in the direction of the axis of rotation.

19. The apparatus according to claim 2,
wherein
the second lifting device is configured to displace a predetermined second portion of the second holding devices relative to a predetermined first portion of the second holding devices in the direction of the axis of rotation.

20. The apparatus according to claim 2,
wherein
the application device has a plurality of application elements which are each assigned to the holding devices.
